(19) United States
(12) Patent Application Publication
(10) Pub. No.: US 2017/0209100 A1
(43) Pub. Date: Jul. 27, 2017

(54) SYSTEMS AND METHODS FOR REDUCING NOISE IN A NEURAL RECORDING DEVICE

(71) Applicant: The Charles Stark Draper Laboratory, Inc., Cambrige, MA (US)

(72) Inventor: Andrew Czarnecki, Cambridge, MA (US)

(21) Appl. No.: 15/008,030

(22) Filed: Jan. 27, 2016

(51) Int. Cl. A61B 5/00; A61B 5/04

(52) U.S. Cl. CPC A61B 5/7203; A61B 5/04001; A61B 5/0004; A61B 5/6877; A61B 2560/0219

(57) ABSTRACT

This disclosure provides systems and methods for reducing noise in neural recording device. A neural recording system can include a radiofrequency (RF) transmitter configured to transmit an RF signal. A neural electrode can be configured to receive a neural signal from nervous tissue. An analog to digital converter (ADC) configured to, receive the neural signal from the neural electrode, sample the received neural signal, and generate a digital output based on the sampled neural signal. The system also can include a phase-locked loop (PLL) oscillator configured to synchronize the sampling frequency of the ADC with the RF signal.

### BACKGROUND

Nervous tissue can produce electrical signals. Measuring and recording these electrical signals from nervous tissue can lead to a better understanding of neural activity, which can aid in characterizing normal neural activity or diagnosing neurological conditions. In some cases, neural recording devices can be implanted adjacent to neural tissue. However, electrical signals produced by neural tissue can have relatively low magnitudes, making them difficult to measure. Furthermore, neural recording devices may be susceptible to noise from the external environment, which may corrupt recorded neural signals.

### SUMMARY

The systems, methods, and devices of the disclosure each have several innovative aspects, no single one of which is solely responsible for the desirable attributes disclosed herein.

One innovative aspect of the subject matter described in this disclosure can be implemented in a neural recording system for recording neural signals in a body. The system can include a radiofrequency (RF) transmitter configured to transmit a first RF signal. The system can include a neural electrode configured to receive a neural signal from nervous tissue. The system can include an analog to digital converter (ADC) configured to receive the neural signal from the neural electrode, sample the received neural signal, and generate a digital output based on the sampled neural signal. The system also can include a phase-locked loop (PLL) oscillator configured to synchronize the sampling frequency of the ADC with the first RF signal.

In some implementations, the PLL oscillator can be further configured to receive the first RF signal as a reference clock input and generate an output clock signal based on the reference input. In some implementations, the PLL oscillator can be further configured to generate the output clock signal such that the output clock signal has a frequency that is an integer factor of a frequency of the first RF signal. In some implementations, the ADC can be further configured to receive the output clock signal from the PLL oscillator, and sample the received neural signal at a frequency matching the frequency of the output clock signal received from the PLL oscillator. In some implementations, the first RF signal can have a frequency in the range of about 60 Hz to about 1 GHz.

In some implementations, the PLL oscillator can include at least one of a tunable crystal oscillator, an LC oscillator, and a ring oscillator. In some implementations, the system also can include a clock buffer configured to process the first RF signal before the first RF signal is delivered to the PLL oscillator. In some implementations, each of the neural electrode, the ADC, and the PLL oscillator can be implanted within the body and the RF transmitter is external to the body. In some implementations, the system also can include a power rectifier configured to receive the first RF signal from the RF transmitter, and convert the first RF signal into a direct current (DC) output for providing power to at least one of the implanted components of the neural recording system.

In some implementations, the system also can include a second RF transmitter configured to receive the digital output from the ADC and transmit a second RF signal corresponding to an encoding of the digital output. The system also can include a receiver external to the body, the receiver configured to receive the second RF signal from the second RF transmitter. In some implementations, the second RF transmitter and the PLL oscillator can be further configured to receive a common reference clock input. The second RF transmitter can be further configured to transmit the second RF signal at a frequency phase locked to the frequency of the common reference clock input. In some implementations, the receiver can be further configured to filter the received encoding of the digital output to remove a component corresponding to interference from the first RF signal and the second RF signal.

Another innovative aspect of the subject matter described in this disclosure can be implemented in a method for recording a neural signal. The method can include receiving, by an implantable neural recording system, a first RF signal. The method can include generating, by the implantable neural recording system, a reference clock signal synchronized to the first RF signal. The method can include sampling, by the implantable neural recording system, a neural signal at a sampling frequency matching a frequency of the generated reference clock signal. The method can include generating, by the implantable neural recording system, a digital output based on the sampled neural signal.

In some implementations, the first RF signal can be configured to supply power to the implantable neural recording system. In some implementations, the method also can include rectifying the first RF signal to generate a direct current (DC) output for use by the implantable neural recording system. In some implementations, the first RF signal can have a frequency in the range of about 60 Hz to about 1 GHz. In some implementations, generating the reference clock signal can include generating the reference clock signal such that the reference clock signal has a frequency that is an integer factor of a frequency of the first RF signal.

In some implementations, the method also can include transmitting, by the implantable neural recording system, a second RF signal corresponding to the digital output. In some implementations, transmitting the second RF signal can include transmitting the second RF signal at a frequency phased locked to a frequency of the first RF signal. In some implementations, the method also can include filtering the digital output to remove a component of the digital output corresponding to interference from the first RF signal and the second RF signal.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar elements, and/or structurally similar elements throughout the various drawings. The drawings are not necessarily to scale. Instead, emphasis is placed upon illustrating the principles of the teachings. The drawings are not intended to limit the scope of the present teachings in any way. The system and method may be better understood from the following illustrative description with reference to the following drawings in which:

FIG. 1 shows a block diagram of an example implantable neural recording system, according to an illustrative implementation.

FIG. 2 shows a block diagram of another example implantable neural recording system, according to an illustrative implementation.

FIG. 3 shows a block diagram of another example implantable neural recording system, according to an illustrative implementation.

FIG. 4 shows a block diagram of an example method for recording a neural signal, according to an illustrative implementation.

Like reference numbers and designations in the various drawings indicate like elements.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

This disclosure is generally related to systems and methods for reducing noise in a neural recording device. More particularly, the systems and methods disclosed provide techniques for reducing the negative effects of electromagnetic interference that may corrupt the analog neural signals received by an implantable neural recording device. In some implementations, such electromagnetic interference may originate with a radiofrequency (RF) transmitter associated with the implantable device, such as a wireless power transmitter or a wireless data transmitter.

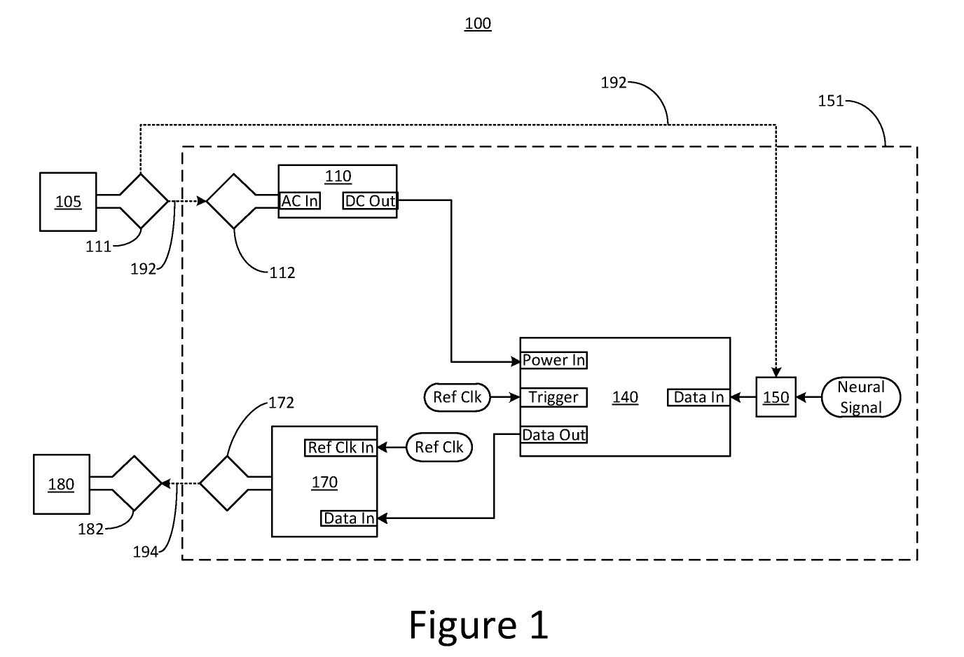

FIG. 1A shows a block diagram of an example implantable neural recording system 100, according to an illustrative implementation. Components of the neural recording system 100 shown inside the box 151 are implanted within the body of a patient, and components shown outside of the box 151 are external to the body. For example, the neural recording system 100 includes an electrode 150, an analog-to-digital converter (ADC) 140, and a power rectifier 110 coupled to an antenna 112, all of which are implanted components. The neural recording system 100 also includes an external wireless RF transmitter 105 coupled to an antenna 111. Additionally, the neural recording system 100 includes a second RF transmitter 170 coupled to an antenna 172, both of which are implanted within the patient. An external antenna 182 coupled to a receiver 180 can be configured to receive a wireless signal 194 transmitted by the second RF transmitter 170.

The implanted components of the neural recording system 100 are powered wirelessly by the external RF transmitter 105. Such a configuration can avoid the inconvenience and health risks associated with having wires protruding from the body of the patient in order to supply power to the implanted components of the neural recording system 100. The RF transmitter 105 can transmit a wireless signal 110 that can be received by the implanted antenna 112. The power rectifier 110 can receive the signal from the antenna 112 via an AC input, and can rectify the received signal to generate a DC power signal. The DC power signal is provided at a DC output of the power rectifier 110, which is coupled to a power input of the ADC 140.

The neural electrode 150 can be implanted in the patient adjacent to nervous tissues, such as a neuron, nerve fiber, or a nerve bundle, and can be configured to receive an analog neural signal produced by the nervous tissue. The analog neural signal received by the electrode 150 is delivered to an input of the ADC 140. The ADC 140 can digitize the received analog neural signal by sampling the analog neural signal at regular intervals. For example, sampling of the analog neural signal can be controlled by a trigger input of the ADC 140, which can be, for example, a reference clock input. In some implementations, the ADC 140 can be configured to collect one sample of the analog neural signal each clock cycle. In some implementations, the frequency of the reference clock input to the ADC 140 can be selected based on an expected frequency of the analog neural signal. For example, the frequency of the reference clock input to the ADC 140 can be selected to be at least twice the expected maximum frequency of the analog neural signal, in order to avoid aliasing the analog neural signal. The digital representation of the analog neural signal generated by the ADC 140 can be provided at a data output of the ADC 140 coupled to the second RF transmitter 170. The second RF transmitter 170 can be configured to receive the digital output from the ADC 140, as well as a reference clock input. The second RF transmitter 170 can transmit an encoding of the received digital data to the external receiver 180 at a frequency that is phase locked to the frequency of the reference clock. In some implementations, the frequency of the signal transmitted by the transmitter 170 may be significantly higher than the frequency of the reference clock input to the transmitter 170. For example, in some implementations, the reference clock input to the transmitter 170 may have a frequency in the range of about 50 kHz to about 200 kHz, and the signal transmitted by the transmitter 170 may have a frequency in the range of about 1 GHz to about 3 GHz. In some implementations, the reference clock input to the transmitter 170 may have a frequency of about 100 kHz and the signal transmitted by the transmitter 170 may have a frequency of about 2.4 GHz.

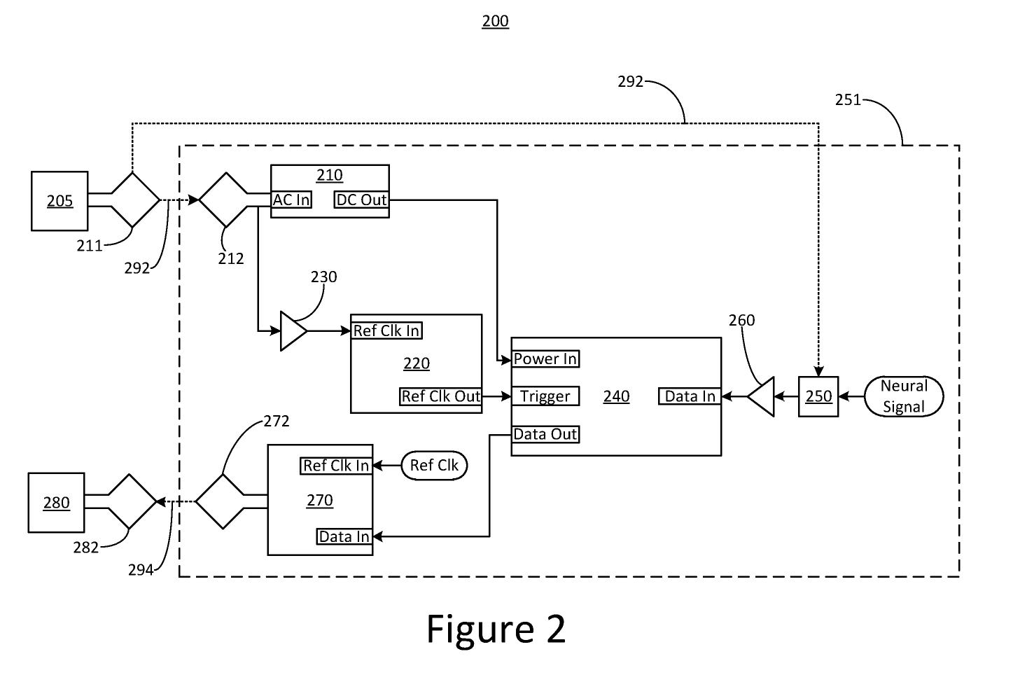

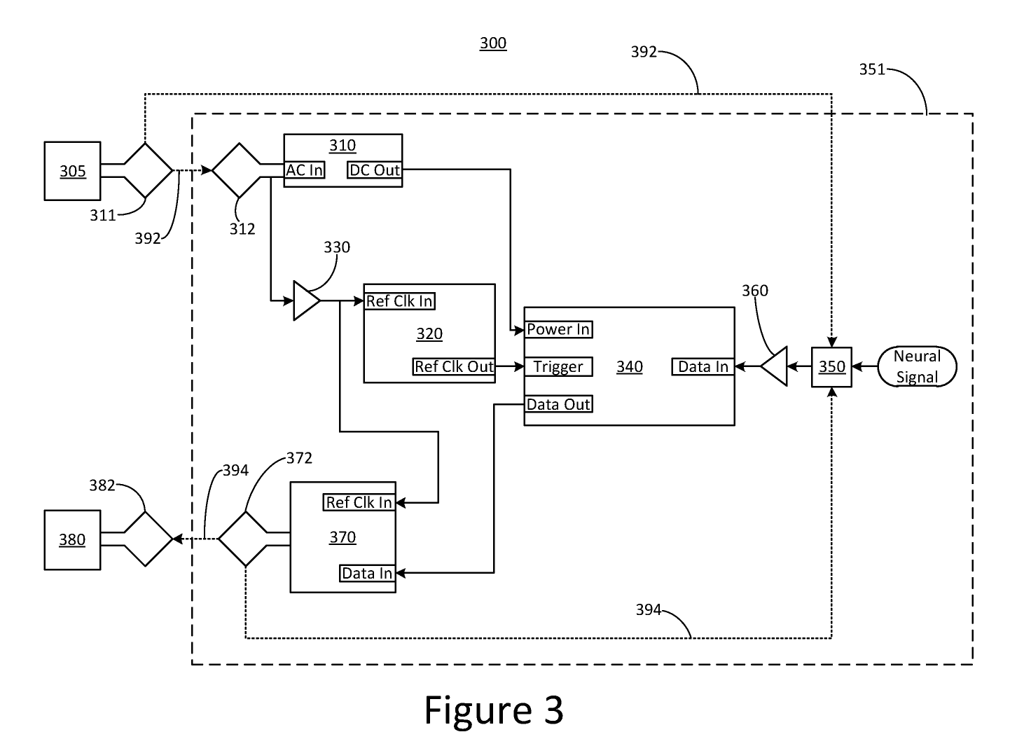

In some implementations, the neural recording system 100 may be susceptible to noise that can prevent the system 100 from generating an accurate digital representation of the analog neural signal. For example, the electrode 150 may also receive interfering signals and, as a result, the analog signal delivered to the ADC 140 may become corrupted. In some implementations, such an interfering signal may originate at the external RF transmitter 105. For example, while the wireless signal 192 transmitted from the RF transmitter 105 is intended only for the power rectifier 110, the wireless signal 192 may be pervasive in the space around all of the implanted components of the neural recording system 100, including the electrode 150. FIG. 1 shows the path of the wireless signal 192 from the antenna 111 of the RF transmitter 105 to the implanted electrode 150. As a result of the interference caused by the wireless signal 192, the signal detected by the electrode 150 is a summation of the analog neural signal and the wireless signal 192. When the ADC 140 receives the signal from the electrode 150, the presence of the interfering wireless signal 192 can prevent the ADC 140 from producing an accurate representation of the analog neural signal. In some cases, the frequency of the wireless signal 192 may be higher than half the sampling rate of the ADC 140 (i.e., higher than half the frequency of the reference clock input to the ADC 140). For example, the frequency of the wireless signal 192 may be in the range of about 50 kHz to about 1 MHz, and the sampling rate of the ADC 140 may in the range of about 10 kHz to about 30 kHz. Sampling by the ADC 140 of an input signal having a frequency higher than half the sampling rate can result in aliasing effects that further corrupt the digital output signal generated by the ADC 140. Other frequencies for the wireless signal 192 are also possible. For example, in some implementations, the wireless signal 192 may have a frequency in the range of about 60 Hz to about 1 GHz. In some implementations, the receiver 180 may include one or more electronic filters configured to attenuate the interfering wireless signal 192, but such filters may not completely reject the interfering wireless signal 192. Techniques for mitigating the effects of interfering signals, such as the wireless signal 192, are described below in connection with FIGS. 2 and 3.

FIG. 2 shows a block diagram of another example implantable neural recording system 200, according to an illustrative implementation. The neural recording system 200 includes many components similar to those described above in connection with the neural recording system 100 shown in FIG. 1, and like reference numerals refer to like components. For example, the neural recording system 200 includes an electrode 250, an ADC 240, and a power rectifier 210 coupled to an antenna 212. All of those components, shown inside the box 251, can be implanted in a patient. The system 200 also includes an external RF transmitter 205 and an antenna 211. Additionally, the neural recording system 200 includes a second RF transmitter 270 coupled to an antenna 272, both of which are also implanted within the patient. An external antenna 282 coupled to a receiver 280 can be configured to receive a wireless signal 294 transmitted by the second RF transmitter 270. To improve the ability of the ADC 240 to reject interference from the wireless signal 292, the neural recording system 200 also includes a phase-locked loop (PLL) oscillator 220.

Operation of the neural recording system 200 is similar to that of the neural recording system 100 shown in FIG. 1. For example, the RF transmitter 205 can transmit a wireless signal 292 that can be received by the implanted antenna 212. The power rectifier 210 can receive the signal from the antenna 212 via an AC input, and can rectify the received signal to generate a DC power signal. The DC power signal is provided at a DC output of the power rectifier 210, which is coupled to a power input of the ADC 240. The analog neural signal received by the electrode 250, which may be corrupted by the wireless signal 292, is passed through an analog amplifier 260 before being delivered to an input of the ADC 240. The ADC 240 can digitize the received analog signal by sampling the analog neural signal at regular intervals.

Sampling of the analog neural signal can be controlled by a trigger input of the ADC 240, which can receive a reference clock input. In some implementations, the ADC 240 can be configured to collect one sample of the analog neural signal each clock cycle. In some implementations, the frequency of the reference clock input to the ADC 240 can be selected based on an expected frequency of the analog neural signal. For example, the frequency of the reference clock input to the ADC 240 can be selected to be at least twice the expected maximum frequency of the analog neural signal, in order to avoid aliasing the analog neural signal. The digital representation of the analog neural signal generated by the ADC 240 can be provided at a data output of the ADC 240. The second RF transmitter 270 can be configured to receive the digital output from the ADC 240, as well as a reference clock input. The second RF transmitter 270 can transmit an encoding of the received digital data to the external receiver 280 at a frequency that is phased locked to the frequency of the reference clock.

The PLL oscillator 220 can help to reduce the effect of interference from the wireless signal 292 by synchronizing the sample clock of the ADC 240 with the signal 292 transmitted by the RF transmitter 205. As shown in FIG. 2, the wireless signal 292 received by the implanted antenna 212 feeds into a reference clock input of the PLL oscillator 220 via a clock buffer 230. The PLL oscillator 220 can be any type of oscillator that is configured to generate a reference clock output based on the received reference clock input. For example, in some implementations, the PLL oscillator 220 can be implemented as a tunable crystal oscillator, an LC oscillator, or a ring oscillator.

In some implementations, the PLL oscillator 220 can be configured to produce a reference clock output having a frequency and a phase that are identical to those of the received reference clock input. In some other implementations, the reference clock output of the PLL oscillator 220 may have a frequency that is an integer factor (or an integer multiple) of the received reference clock input, and a phase that is the same as that of the reference clock input. The output of the PLL oscillator 220 feeds into the trigger output of the ADC 240, which is used to trigger sampling of the analog signal received from the electrode 250, as described above.

The above configuration can ensure that the sampling of the ADC 240 is phase locked to the frequency of the wireless signal 292. As a result, certain forms of noise such as drift in the frequency of the interfering wireless signal 292 can be coherently replicated at the trigger input to the ADC 240. Because such noise is coherently replicated, the sampling process used by the ADC 240 will cancel it out without the need for any additional filtering. Other types of noise, such as amplitude drift of the interfering wireless signal 292, may still interfere with the operation of the ADC 240, but the synchronization of the wireless signal 292 with the trigger input to the ADC 240 can simplify the process of filtering such noise. For example, if the wireless signal 292 has a frequency that is greater than half the sampling rate of the ADC 240, aliasing will occur and will result in a corrupted output signal. However, if the sampling rate of the ADC 240 is an integer factor of the frequency of the wireless signal 292, the aliasing effect will result only in a DC offset, which can be relatively easy to filter, for example by using a high-pass filtering algorithm in the digital domain. The PLL oscillator 220 can ensure that the sampling rate of the ADC 240 is an integer factor of the frequency of the wireless signal 292, thereby reducing the complexity of removing the aliasing effects from the digital output of the ADC 240. As a result, the receiver 280 can employ relatively simple electronic filtering techniques to remove noise caused by the interfering wireless signal 292 from the data it receives from the ADC 240.

FIG. 3 shows a block diagram of another example implantable neural recording system 300, according to an illustrative implementation. The neural recording system 300 includes many components similar to those described above in connection with the neural recording system 200 shown in FIG. 2, and like reference numerals refer to like components. For example, the neural recording system 200 includes an electrode 350, an analog amplifier 360 an ADC 340, a power rectifier 310 coupled to an antenna 312, a clock buffer 330, and a PLL oscillator 320. All of those components, shown inside the box 351, can be implanted in a patient. The system 300 also includes an external RF transmitter 305 and an antenna 311. Additionally, the neural recording system 300 includes a second RF transmitter 370 coupled to an antenna 372, both of which are also implanted within the patient. An external antenna 382 coupled to a receiver 380 can be configured to receive a wireless signal 394 transmitted by the second RF transmitter 370. The second RF transmitter 370 can be configured to receive a digital output from the ADC 340 and transmit the received digital data to the external receiver 380.

The wireless signal 392 transmitted by the RF transmitter 305 can interfere with the operation of the neural recording system 300 in a manner similar to that discussed above in connection with the neural recording system 200 of FIG. 2, and the PLL oscillator 300 can also help to mitigate the effects of such interference in a manner similar to the PLL oscillator 200 shown in FIG. 2. However, in the neural recording system 300, the wireless signal 394 transmitted by the second RF transmitter can act as an additional source of interference that can lead to corruption of the signal received by the neural electrode 350. As shown, although the wireless signal 394 is intended to be transmitted to the receiver 380, some components of the wireless signal 394 also may be unintentionally received by the neural electrode 350.

To mitigate the interference from the wireless signal 394, the second RF transmitter 370 can be phase-locked to the sample clock of the ADC 340. For example, the second RF transmitter 370 can include a clock input that receives the same signal received at the reference clock input of the PLL oscillator 320. As discussed above, the PLL oscillator 320 generates a clock output that is phase-locked to its reference clock input, and the ADC 340 is configured to collect samples of the signal received from the electrode 350 at intervals controlled by the clock output of the PLL oscillator 320. Thus, the second RF transmitter 370 can receive a reference clock input that is phase-locked to the sample clock of the ADC 340, and can be configured to transmit the wireless signal 394 at a frequency corresponding to the frequency of its reference clock input. As a result, the sampling of the ADC 340 is phase locked to the frequency of the wireless signal 394, and certain forms of noise caused by the wireless signal 394 can be easily removed from the output of the ADC 340 in a manner similar to the removal of the noise caused by the wireless signal 392.

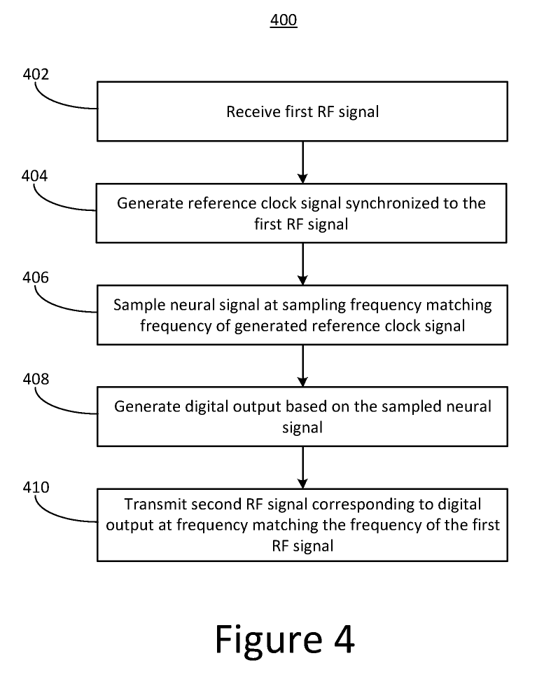

FIG. 4 shows a block diagram of an example method 400 for recording a neural signal, according to an illustrative implementation. In brief overview the method 400 includes receiving a first RF signal (stage 402). A reference clock signal synchronized to the first RF signal is generated (stage 404). A neural signal is sampled at a sampling frequency matching a frequency of the generated reference clock signal (stage 406). A digital output is generated based on the sampled neural signal (stage 408). A second RF signal corresponding to the digital output can then be transmitted at a frequency that is phased locked the frequency of the first RF signal (stage 410).

In some implementations, the method 400 can be carried out by the implantable neural recording systems 200 and 300 shown in FIGS. 2 and 3, respectively. For example, with reference to FIGS. 3 and 4, the rectifier 310 can receive an RF signal 392 from the transmitter 305 (stage 402). The PLL oscillator 320 can generate a reference clock signal synchronized to the RF signal 392 (stage 404). The ADC 340 can sample a neural signal at a sampling frequency matching the frequency of the reference clock output of the PLL oscillator 320 (stage 406), for example by triggering the collection of one sample of the neural signal for each clock cycle of the reference clock. The ADC 340 can generate a digital output based on the sampled neural signal (stage 408), and this digital output can be provided to the second RF transmitter 370. The second RF transmitter 370 also can receive a reference clock input having the same frequency as the frequency of RF signal 392. As a result, the second RF transmitter 370 transmits a second RF signal 394 corresponding to the digital output of the ADC 340 at a frequency phased locked to the frequency of the first RF signal 392 (stage 410).

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations. Additionally, other implementations are within the scope of the following claims. In some cases, the actions recited in the claims can be performed in a different order and still achieve desirable results.

What is claimed is:

1. A neural recording system for recording neural signals in a body, the system comprising:
a radiofrequency (RF) transmitter configured to transmit a first RF signal;
a neural electrode configured to receive a neural signal from nervous tissue;
an analog to digital converter (ADC) configured to:
receive the neural signal from the neural electrode;
sample the received neural signal; and
generate a digital output based on the sampled neural signal; and
a phase-locked loop (PLL) oscillator configured to synchronize the sampling frequency of the ADC with the first RF signal.

2. The neural recording system of claim 1, wherein the PLL oscillator is further configured to:
receive the first RF signal as a reference clock input; and
generate an output clock signal based on the reference input.

3. The neural recording system of claim 2, wherein the PLL oscillator is further configured to generate the output clock signal such that the output clock signal has a frequency that is an integer factor of a frequency of the first RF signal.

4. The neural recording system of claim 2, wherein the ADC is further configured to:
receive the output clock signal from the PLL oscillator; and
sample the received neural signal at a frequency matching the frequency of the output clock signal received from the PLL oscillator.

5. The neural recording system of claim 1, wherein the first RF signal has a frequency in the range of about 60 Hz to about 1 GHz.

6. The neural recording system of claim 1, wherein the PLL oscillator comprises at least one of a tunable crystal oscillator, an LC oscillator, and a ring oscillator.

7. The neural recording system of claim 1, further comprising a clock buffer configured to process the first RF signal before the first RF signal is delivered to the PLL oscillator.

8. The neural recording system of claim 1, wherein each of the neural electrode, the ADC, and the PLL oscillator is implanted within the body and the RF transmitter is external to the body.

9. The neural recording system of claim 8, further comprising a power rectifier configured to:
receive the first RF signal from the RF transmitter; and
convert the first RF signal into a direct current (DC) output for providing power to at least one of the implanted components of the neural recording system.

10. The neural recording system of claim 1, further comprising:
a second RF transmitter configured to:
receive the digital output from the ADC; and
transmit a second RF signal corresponding to an encoding of the digital output; and
a receiver external to the body, the receiver configured to receive the second RF signal from the second RF transmitter.

11. The neural recording system of claim 10, wherein the second RF transmitter and the PLL oscillator are further configured to receive a common reference clock input, and wherein the second RF transmitter is further configured to transmit the second RF signal at a frequency phase locked to the frequency of the common reference clock input.

12. The neural recording system of claim 10, wherein the receiver is further configured to filter the received encoding of the digital output to remove a component corresponding to interference from the first RF signal and the second RF signal.

13. A method for recording a neural signal, the method comprising:
receiving, by an implantable neural recording system, a first RF signal;
generating, by the implantable neural recording system, a reference clock signal synchronized to the first RF signal;
sampling, by the implantable neural recording system, a neural signal at a sampling frequency matching a frequency of the generated reference clock signal; and
generating, by the implantable neural recording system, a digital output based on the sampled neural signal.

14. The method of claim 13, wherein the first RF signal is configured to supply power to the implantable neural recording system.

15. The method of claim 14, further comprising rectifying the first RF signal to generate a direct current (DC) output for use by the implantable neural recording system.

16. The method of claim 13, wherein the first RF signal has a frequency in the range of about 60 Hz to about 1 GHz.

17. The method of claim 13, wherein generating the reference clock signal further comprises generating the reference clock signal such that the reference clock signal has a frequency that is an integer factor of a frequency of the first RF signal.

18. The method of claim 13, further comprising transmitting, by the implantable neural recording system, a second RF signal corresponding to the digital output.

19. The method of claim 18, wherein transmitting the second RF signal comprises transmitting the second RF signal at a frequency phased locked to a frequency of the first RF signal.

20. The method of claim 19, further comprising filtering the digital output to remove a component of the digital output corresponding to interference from the first RF signal and the second RF signal.
